(19) United States
(12) Patent Application Publication
(10) Pub. No.: US 2014/0073902 A1
(43) Pub. Date: Mar. 13, 2014

(54) MEDICAL IMAGING UNIT WITH A SENSOR UNIT FOR DETECTING A PHYSIOLOGICAL SIGNAL AND METHOD FOR DETECTING A PATIENT'S CARDIAC CYCLE

(71) Applicant: Stefan Popescu, Erlangen (DE)

(72) Inventor: Stefan Popescu, Erlangen (DE)

(21) Appl. No.: 14/021,088

(22) Filed: Sep. 9, 2013

(30) Foreign Application Priority Data
Sep. 13, 2012 (DE) 102012216248.8

(51) Int. Cl. A61B 6/00

(52) U.S. Cl. CPC A61B 6/541
USPC 600/407; 600/508; 600/509; 600/513; 600/485; 600/526

(57) ABSTRACT

A medical imaging device includes a sensor unit for detecting a physiological signal, and a data evaluation unit which, on the basis of the detected physiological signals, determines a trigger signal for a medical imaging examination on a patient. The sensor unit includes at least one sensor element which is designed for detection of at least one blood circulation signal.

### CROSS REFERENCE TO RELATED APPLICATIONS

This application claims priority of German Patent Office application No. 102012216248.8 DE filed Sep. 13, 2012. All of the applications are incorporated by reference herein in their entirety.

### FIELD OF INVENTION

The present invention relates to a medical imaging unit with a sensor unit for detecting a physiological signal and a data evaluation unit which, on the basis of the detected physiological signal, determines a trigger signal for a medical imaging examination on a patient.

### BACKGROUND OF INVENTION

In medical imaging examination the problem frequently occurs that an EKG signal must be detected in order to obtain and/or to generate a trigger signal for a medical imaging measurement, for example a magnetic resonance measurement and/or a computed tomography measurement etc. EKG electrodes are arranged for this purpose on a chest area of the patient. A medical imaging measurement of the chest area of the patient is often performed at the same time. However the medical imaging measurement can adversely affect the detection of the EKG signal and/or conversely the detection of the EKG signal can adversely affect the medical imaging measurement.

In addition the problem frequently occurs here that the arrangement of the EKG unit and/or the arrangement of individual EKG electrodes of the EKG unit can collide with an arrangement of accessory units for the current medical imaging examination. For example the arrangement of the EKG unit can prevent a positioning of a chest coil for a magnetic resonance examination because of a restricted amount of space available on the patient's chest.

Furthermore the arrangement and/or positioning of the EKG electrodes can be especially complex in respect of time, since the EKG electrodes must be arranged directly on the skin of the patient, wherein only a contact gel can be arranged between the EKG electrodes and the skin of the patient. This however requires an increased effort during a preparation of the patient for the imminent medical imaging examination. Thus for example the skin area of the patient intended for the arrangement of the EKG electrodes must be shaved beforehand by the operating personnel.

### SUMMARY OF INVENTION

The particular object underlying the present invention is to provide a medical imaging device and a method in which an especially simple and time-saving arrangement of the sensor elements is made possible. This object is achieved by the features of the independent claims. Advantageous embodiments are described in the subclaims

The invention is based on a medical imaging device with a sensor unit for detecting a physiological signal and a database evaluation unit which, on the basis of the detected physiological signal, determines a trigger signal for a medical imaging examination on a patient.

It is proposed that the sensor unit has at least one sensor element which is designed to detect at least one blood circulation signal. This enables a workflow for clinical operating personnel to be simplified since the sensor unit and/or the sensor element can be arranged in an area of the patient in which no further additional units for the medical imaging examination are arranged. In particular time-consuming preparation of the patient can be dispensed with for the clinical operating personnel such as for example undressing the patient and/or preparation of an area of skin for an arrangement of EKG electrodes etc., since advantageously a measurement by means of an EKG unit for detecting a physiological signal can be dispensed with. In this context a blood circulation signal is especially to be understood as the signal which for example is formed by a blood pressure signal and/or a pulse signal of the patient etc. However a blood circulation signal is especially to be understood as not being any signal which is detected by an EKG unit.

Especially advantageously the at least one sensor element for a detection of the blood circulation signal is disposed on the patient's arm and/or leg and/or neck, so that especially a chest area of the patient is available for arrangement of further units especially of coil units, for example a chest coil for a magnetic resonance examination. In this way in an undesired adverse affect on the medical imaging measurement by the detection of the blood circulation signal can additionally be prevented, since the blood circulation signal can be detected at an area of the patient which is not relevant to the medical imaging measurement. Furthermore the blood circulation signal can also be detected undisturbed during the medical imaging measurement.

An especially cost-effective sensor unit can be achieved if the at least one sensor element comprises a pulse sensor element and/or a blood pressure sensor element, by means of which a blood pressure signal and/or a pulse signal are able to be detected. Preferably the blood pressure signal and/or the pulse signal has a direct correlation with the cardiac cycle of the patient, so that by means of the blood pressure signal and/or the pulse signal a cardiac cycle of the patient can be deduced directly in an especially simple manner and in this way a trigger signal for the medical imaging examination can be detected and/or determined

In an advantageous development of the invention it is proposed that the at least one sensor element comprises an optical sensor element. In this way the blood circulation signal, especially a blood pressure signal and/or a pulse signal, can be detected especially quickly and especially without direct contact between the sensor element and the patient.

The optical sensor element can in such cases include a laser unit and a laser detector unit, wherein the laser unit emits a laser signal and the laser detector unit detects the laser signal reflected on the patient. An advantageous focusing of the optical sensor element on an area of the patient can be achieved by means of the laser unit, which is advantageous for detecting the blood circulation signal, such as a patient's arm and/or neck for example.

In a further embodiment of the invention it is proposed that the sensor unit includes at least two first sensor elements and at least two second sensor elements, wherein a voltage is able to be detected between the at least two further sensor elements during a contact with the patient and an alternating current flowing between the at least two first sensor elements. A blood circulation signal of the patient can be advantageously detected since the voltage present at the at least two further sensor elements and/or an electrical impedance present at the at least two further sensor elements is dependent on a physiology, especially on a blood flow and/or blood circulation of the patient. In this way a signal curve correlated with the cardiac cycle of the patient and/or a possible EKG signal of the patient can be detected.

In addition it is proposed that the sensor unit has a data transmission unit with at least one antenna element, through which advantageously a wireless and/or cordless data transmission between for example the sensor unit and a data evaluation unit and/or a control unit of the medical imaging device and/or further units appearing sensible to the person skilled in the art can be achieved. The antenna element also enables additional cables and/or data lines for detecting and/or forwarding of the physiological signals, especially the blood circulation signals, to be dispensed with and in this way a workflow for preparing the patient for the medical imaging examination to be advantageously simplified.

In an advantageous development of the invention it is proposed that a reference signal is able to be detected by means of the sensor unit, wherein the reference signal is embodied differently to the physiological signal, especially the blood circulation signal. This enables a correction value, especially a time delay to a cardiac cycle of the patient, to be determined for the detected physiological signal, especially blood pressure signal and/or pulse signal, so that an ideal trigger point can be determined on the basis of the physiological signal. The reference measurement in this case can be undertaken before the detection of the physiological signal or the medical imaging examination. Preferably the physiological signal here comprises a pulse signal and the reference signal a blood pressure signal.

In addition there can be provision for a further reference signal to be detected during the duration of the medical imaging measurement, so that a correction value for the detected physiological signal can always be adapted to the current state and/or to a current diagnosis of the patient. This enables an exact and/or ideal trigger time for individual measurement segments of the medical imaging examination to always be achieved.

As an alternative to this the medical imaging device can also comprise a further sensor unit, especially a reference signal sensor unit, by means of which a reference signal is able to be detected. The further sensor unit can for example comprise an EKG unit so that the physiological sensor signal can be directly compared with an EKG signal as reference signal, wherein the EKG signal or the reference measurement for detecting the EKG signal is preferably detected before the medical imaging examination begins. In addition there can be provision for the further sensor unit to comprise a blood pressure sensor unit.

Furthermore the further sensor unit can be embodied at least in some cases in one piece with the detector unit, which enables an especially space-saving embodiment of the medical imaging device to be achieved. The reference measurement can for example be made by means of a navigator measurement of the medical imaging device, for example of a magnetic resonance device, through which advantageously a trigger signal for especially individual measurement segments of the medical imaging examination can be determined and thus a measurement synchronized with the cardiac cycle of the patient can be performed.

The invention is further based on a method for detecting a cardiac cycle of a patient for generating a trigger signal for a medical imaging examination on the patient, with the following method steps:

A detection of a physiological signal, especially of a first blood circulation signal at the patient,

A determination of a correction value for the physiological signal,

An evaluation of the physiological signal together with the correction value, wherein a time delay of the physiological signal to the cardiac cycle of the patient is determined by means of the correction value, and

Generation of the trigger signal as a function of the detected physiological signal and the determined time delay by means of a data evaluation unit.

By means of the physiological signal and the correction value, this enables an especially exact determination of a trigger point for the medical imaging examination on the patient to be achieved. In addition a workflow for clinical operating personnel can be simplified, since the sensor unit and/or the sensor element can be disposed in an area of the patient in which no further additional units for the medical imaging examination are disposed. In particular a time-consuming preparation of the patient for the clinical operating personnel can be dispensed with, such as for example undressing the patient and/or preparation of an area of skin for the EKG electrodes etc., since advantageously a measurement by means of an EKG unit for detecting a physiological signal can be dispensed with.

Furthermore it is proposed that the correction value is read out from a database, by which especially fast access to the correction value for the evaluation of the physiological signal can be achieved. Preferably the database comprises a lookup table in which the correction values are stored. The individual correction values can for example be read out from the database as a function of a measured pulse signal, wherein the correction value read out from the database can include a time delay of the pulse signal to an actual cardiac cycle of the patient. Preferably the correction value, determined once, is the same for the entire medical imaging examination.

It is further proposed that a reference signal be detected at the patient for determining the correction value. This enables the correction value to be detected especially exactly. In particular here the correction value can be detected and/or determined as a function of the cardiac cycle of the patient and/or as a function of a blood pressure signal of the patient etc. Preferably the correction value is calculated from the reference signal.

An especially simple detection of the reference signal, in which an overlap and/or an undesired influence on the detection of the physiological signal and/or on the medical imaging examination can be prevented, can be achieved when the reference signal is detected before the medical imaging measurement.

In addition it is also possible for the reference signal to be detected during the medical imaging measurement, so that a current reference signal and/or a current correction value for correcting the physiological signal is always available during the medical imaging measurement. Preferably in this case the correction value and thus also the generated trigger signal are adapted to the current cardiac cycle of the patient.

The reference signal can for example be an EKG signal and/or a blood pressure signal, through which an especially simple dependency of the physiological signal, especially of the pulse signal, on the reference signal can be directly detected. For example the EKG signal is detected before the medical imaging examination, wherein the patient is in a waiting room here. An EKG sensor mat, which is arranged on a chair or a couch in the waiting room, can additionally be used for EKG signal detection.

It is further proposed that the reference signal includes a signal of a navigator measurement of the medical imaging device, for example a magnetic resonance device. In this case especially advantageously an additional sensor unit for detecting the reference signal can be dispensed with, through which an especially simple workflow for medical operating personnel can be achieved.

In a further embodiment of the invention it is proposed that a further blood circulation signal, which is different from the first blood circulation signal, is detected at least sometimes during the medical imaging examination and that a change of the time delay to the cardiac cycle of the patient is determined as a function of the further blood circulation signal. This enables the trigger signal to be adapted especially exactly to a cardiac cycle of the patient during the medical imaging examination. In addition this allows an especially fast reaction to changes in the cardiac cycle of the patient during the generation of the trigger signal.

### BRIEF DESCRIPTION OF THE DRAWINGS

Further advantages, features and details of the invention will emerge from the exemplary embodiments described hereinbelow as well as with reference to the drawings,

In the figures:

FIG. 1 shows an inventive medical imaging device with a sensor unit in a schematic diagram,

FIG. 2 shows a section through a first sensor unit,

FIG. 3 shows a section through a second sensor unit,

FIG. 4 shows a diagram of a change in a movement of a skin surface,

FIG. 5 shows a schematic principle of a third sensor unit,

FIG. 6 shows a diagram of a change in an electrical voltage,

FIG. 7 shows an inventive method, and

FIG. 8 shows an alternate method to FIG. 7.

### DETAILED DESCRIPTION OF INVENTION

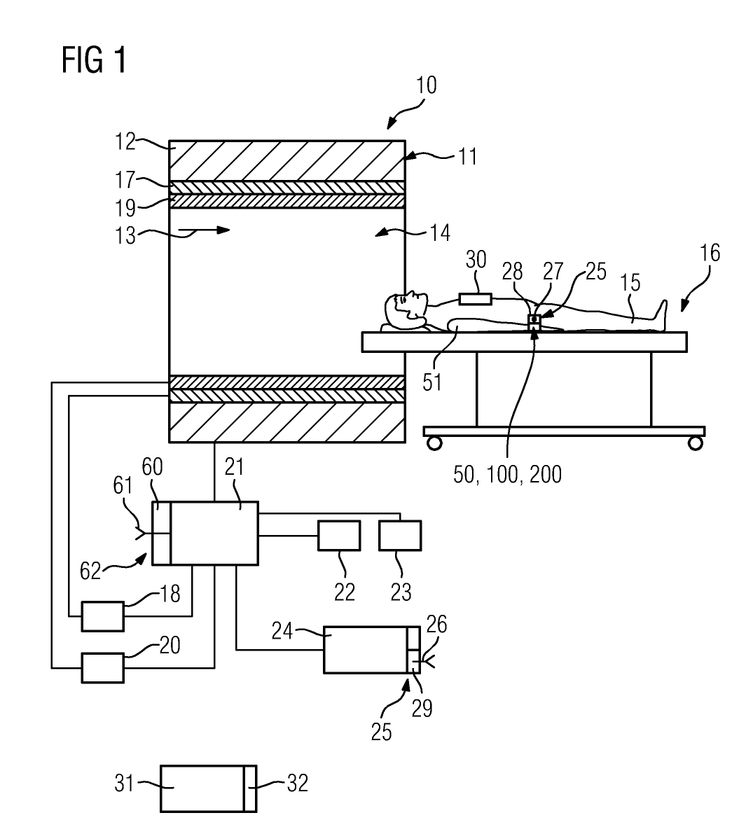

FIG. 1 shows a schematic diagram of an inventive medical imaging device 10 which is formed by a magnetic resonance device. In an alternate embodiment of the medical imaging device 10, this can be formed by a computed tomography device and/or a PET (Positron Emission Tomography) device and/or an AX arm device and/or a SPECT device etc.

The magnetic resonance device comprises a magnet unit 11 having a main magnet 12 for generating a strong and especially constant main magnetic field 13. The magnetic resonance device also has a cylinder-shaped receiving area 14 for receiving a patient 15, the receiving area 14 being enclosed by the magnet unit 11 in a circumferential direction. The patient 15 can be introduced into the receiving area 14 by means of a patient support device 16 of the magnetic resonance device. For this purpose the patient support device 16 is arranged so as to be movable within the magnetic resonance device.

The magnet unit 11 additionally has a gradient coil unit 17 for generating magnetic field gradients, which is used for spatial encoding during an imaging session. The gradient coil unit 17 is controlled by means of a gradient coil control unit 18. The magnet unit 11 also has a radio-frequency antenna unit 19 and a radio-frequency antenna control unit 20 for stimulating a polarization which becomes established in the main magnetic field 13 generated by the main magnet 12. The radio frequency antenna unit 19 is controlled by the radio-frequency antenna control unit 20 and radiates radio-frequency electromagnetic fields into an examination area, which is essentially formed by the receiving area 14. In addition the magnetic resonance device has a local magnetic resonance coil device 30, which is formed in the present embodiment by a chest coil device.

For control of the main magnet 12, the gradient coil control unit 18 and for control of the radio frequency antenna control unit 20, the magnetic resonance device has a control unit 21 formed by a processing unit. The control unit 21 controls the magnetic resonance apparatus centrally, such as the execution of a predetermined imaging gradient echo sequence for example. Control information such as imaging parameters for example, as well as reconstructed magnetic resonance images, can be displayed on a display unit 22, for example on at least one monitor of the magnetic resonance apparatus for an operator. In addition the magnetic resonance apparatus has an input unit 23, by means of which the information and/or parameters can be entered during a measurement process by an operator.

Furthermore the magnetic resonance device has a sensor unit 50, 100, 200 for detecting a physiological signal, wherein the sensor unit 50, 100, 200 is designed to detect a physiological signal formed by a blood circulation signal. On the basis of the detected physiological signal a trigger signal for a magnetic resonance examination on the patient 15 is generated in an evaluation unit 24. The detected physiological signal is transmitted via a data transmission unit 25 of the magnetic resonance device from the sensor unit 50, 100, 200 to the data evaluation unit 24. The data transmission unit 25 has a number of antenna elements 26, 27 for this purpose for cordless and/or wireless data transmission. The sensor unit 50 in this case has a data sending unit 28 of the data transmission unit 25 with the first antenna elements 27 and the data evaluation unit 24 has a data receiving unit 29 of the data transmission unit 25 with the second antenna elements 26.

The trigger signal generated by the data evaluation unit 24 is transmitted by means of a data transmission unit not shown in any greater detail from the data evaluation unit 24 to the control unit 21. As an alternative to this the data evaluation unit 24 can also be integrated within the control unit 21.

Furthermore the magnetic resonance device has a further sensor unit, by means of which a reference signal for the physiological signal, especially the blood circulation signal, is able to be detected. The further sensor unit is formed by a reference signal sensor unit 31. The reference signal sensor unit 31 is formed here by an EKG unit. The reference signal is determined by means of the EKG unit before the magnetic resonance examination, by means of which a correction value, especially a time delay of the blood circulation signal to the cardiac cycle of the patient is determined. As an alternative to the EKG unit the reference signal sensor unit 30 can also be included in the magnet unit, wherein a reference signal measurement can for example be formed by a navigation measurement of the magnet unit before the magnetic resonance examination begins. As an alternative to this the reference signal sensor unit can also be formed by a blood pressure sensor unit etc.

The reference signal sensor unit 31 also includes a data transmission unit 32 with an antenna element.

The magnetic resonance device shown can of course include further components which magnetic resonance devices usually have. A general method of operation of the magnetic resonance device is also known to persons skilled in the art, so that the general components will not be described in any greater detail here.

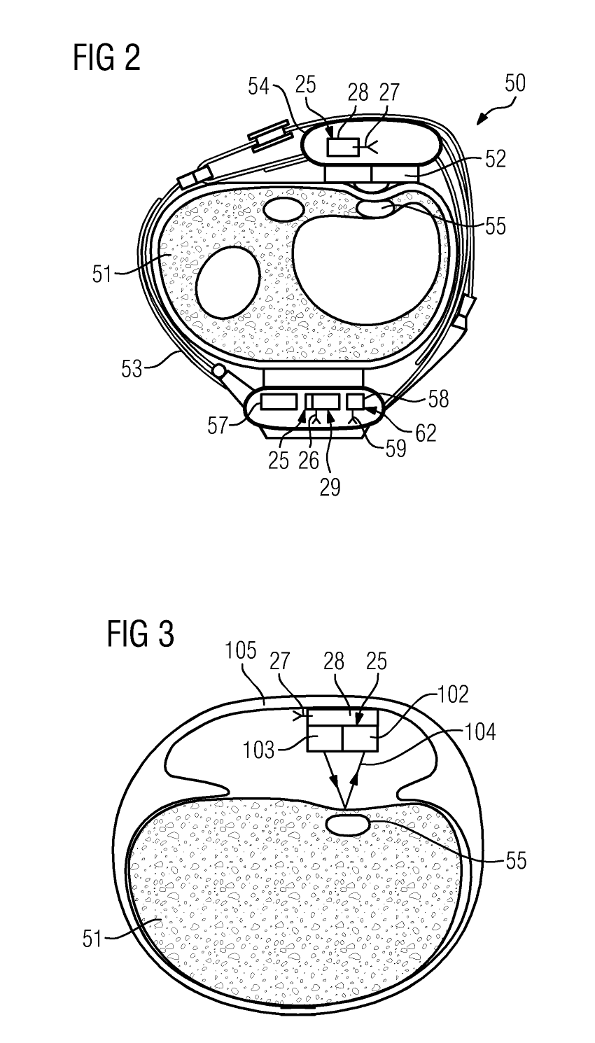

FIG. 2 shows a first embodiment of an sensor unit 50 in greater detail. For detecting the physiological signals the sensor unit is disposed on an arm 51 of the patient 15, especially on a wrist of the patient 15. As an alternative or in addition the sensor unit 50 can also be disposed on the neck and/or leg of the patient 15. The arrangement of the sensor unit 50 on the arm 51, especially on the wrist, of the patient 15 is explained in greater detail here by way of example. An arrangement of the sensor unit 50 on a leg or on the neck of the patient 15 is undertaken in a similar manner to the description for the arrangement on the arm 51 of the patient 15.

The sensor unit 50 is formed by a pulse sensor unit and has a sensor element formed by a pulse sensor element 52 which is designed for detecting a blood circulation signal. The blood circulation signal is formed here by a pulse signal. As well as the pulse sensor element 52, the sensor element 50 comprises an attachment strap 53 by means of which the pulse sensor element 52 is attached to the arm 51 of the patient 15. In this case the sensor unit 50 is attached by means of the attachment strap 53 to the arm 51 of the patient 15 such that the pulse sensor element 52 rests against an inner area of the arm 51, wherein the pulse sensor element 52 rests against the area of the arm 51 covering an artery of the patient 15, especially the Arteris radialis 55 (radial artery). In addition the pulse sensor element 52 is attached to the arm 51 of the patient 15 by means of the attachment strap 53 such that pinching of the Nervus radialis (radial nerve) is prevented and furthermore unrestricted blood flow through the veins disposed in the underarm of the patient 15 can be guaranteed.

The pulse sensor element 52 is disposed within a housing 54 of the sensor unit 50. Furthermore in the present exemplary embodiment the sensor unit 50 already features the data evaluation unit 57, which is likewise disposed by means of the attachment strap 53 on the arm 51 of the patient 15. In this case the pulse sensor element 52 and the data evaluation unit 57 will be disposed in separate positions from one another on the arm 51 of the patient 15 so that a data transmission between the pulse sensor element 52 and the data evaluation unit 57 is undertaken by means of antenna elements 26, 27 of the data transmission unit 57.

Furthermore evaluated or computed data evaluated by the data evaluation unit 57 is transferred via a further data transmission unit 62 between the data evaluation unit 57 and the control unit 21 of the magnetic resonance device. To this end the data evaluation unit 57 has a data sending unit 58 of the further data transmission unit 62 with an antenna element 59. Furthermore the control unit 21 also has a data receiving unit 60 of the further data transmission unit 62 with an antenna element 61. As an alternative to this the data evaluation unit 57 can also be disposed outside the sensor unit 50. In the present exemplary embodiments the data transmission unit 24 is embodied separate to the control unit 21.

The pulse sensor element 52 can be formed in such cases by a conventional, already known pulse sensor element 52, by means of which, in operation or during the magnetic resonance examination, a pulse signal formed by a pulse wave can be detected in a conventional manner non-invasively and exactly. On the basis of the detected pulse signals a heart rate and/or a heart frequency of the cardiac cycle of the patient 15 is determined and/or established by the data evaluation unit 57.

Within the data evaluation unit 57 the correction value, especially the time delay to the cardiac cycle, is also computed and from this a trigger signal for the imminent imaging medical examination is generated.

By means of the pulse sensor element 52 a blood pressure signal can also be established in addition to the pulse signal. As an alternative to this there can also be provision for the sensor unit 50 to have an extra sensor element, especially a blood pressure sensor element, for this purpose. The blood pressure signal can be detected by means of the pulse sensor element 52 before the medical imaging examination. In addition a detection of the blood pressure signal, especially a diastolic and a systolic blood pressure value of the patient 15, is possible simultaneously during the medical imaging examination and during the detection of the pulse signal. The blood pressure signal here is formed by a reference signal which is different from the pulse signal. The blood pressure signal is detected during the entire magnetic resonance measurement in this case so that an adaptation of the correction value, especially the time delay to the cardiac cycle of the patient 15, can be continuously determined.

In addition there can be provision for a diastolic and a systolic blood pressure value of the patient 15 to be determined on the basis of the detected pulse signals by the data evaluation unit 57, so that by means of the pulse sensor element and the detected pulse waves both a signal for detecting a cardiac cycle and/or a heart frequency of the patient 15 and also for a detection of a reference signal for adaptation of the detected pulse signal to the cardiac cycle is present.

FIG. 3 shows a sensor unit 100 embodied alternately to FIG. 2 for detecting a blood circulation signal. The sensor unit 100 is formed by an optical sensor unit and comprises a sensor element formed by an optical sensor element 101, which comprises a laser unit 102 and a laser detector unit 103 for non-invasive detection of the blood circulation signal. The sensor unit 100 has an attachment strap 105, by means of which the sensor unit 100 can be disposed spaced away from the arm 51 of the patient 15 on the arm 51.

A laser signal 104 is generated and sent out by the laser unit 102, whereby the laser signal 104 is directed to an area of the arm 51, including the underarm artery or radial artery, especially Ateria radialis 55 of the patient 15. The laser signal 104 is reflected on the arm 51 of the patient 15 and detected by the laser detector unit 103. Because of unevennesses of the skin surface an optical speckle pattern is created in such cases. This speckle pattern correlates with a blood flow through the artery, wherein the blood flow creates a vibration of the skin area which covers the artery which is imaged in the speckle pattern. These vibrations are also dependent on a blood viscosity which is proportional to a glucose concentration in the blood. Furthermore these vibrations are proportional to a heart rate and/or the cardiac cycle of the patient.

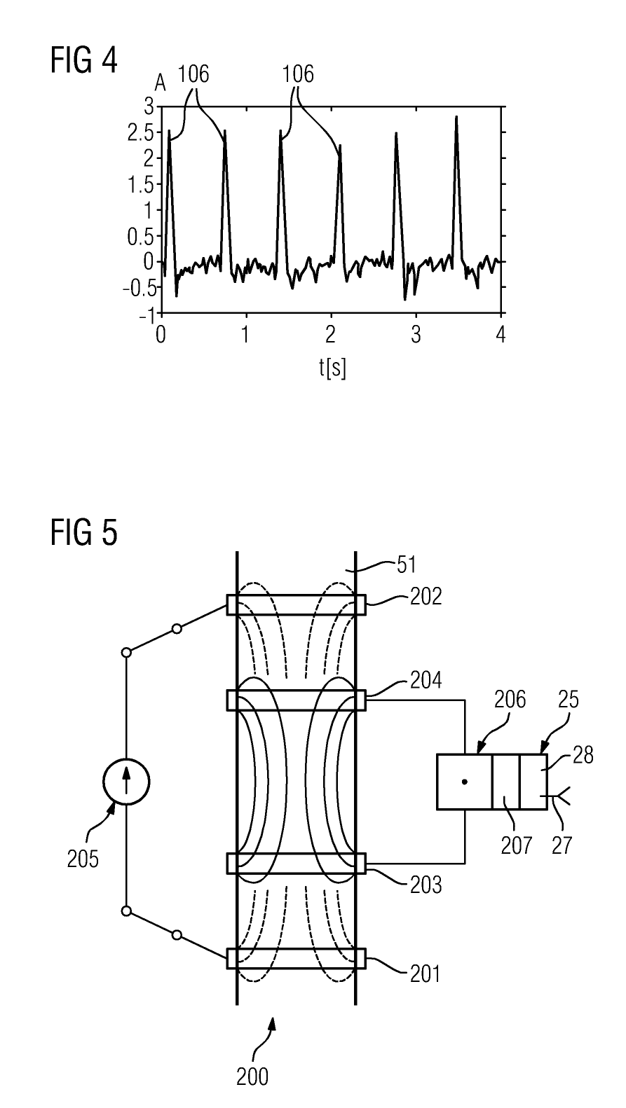

In FIG. 4 an amplitude A of the vibrations or of the speckle pattern is plotted over the time t, wherein in the plotting of the amplitude A, a number of peaks or amplitude maxima 106 repeating at constant intervals can be seen, which correlate with a heartbeat of the patient 15.

The data detected by the optical sensor unit is transmitted by means of the data sending unit 28 with the antenna elements 27 of the data transmission unit 25 to the data evaluation unit 24, wherein the data evaluation unit 24 is embodied separately from the optical sensor unit (FIG. 1).

As can be seen in FIG. 3, the optical sensor unit likewise has an attachment strap, by means of which the optical sensor unit can be attached to the arm 51 of the patient 15. The strap can be attached and disposed here in a manner similar to that described for FIG. 2.

FIG. 5 shows a further exemplary embodiment of the sensor unit 200 for detecting a physiological signal, especially a blood circulation signal. The sensor unit 200 has two first sensor elements 201, 202 and two further sensor elements 203, 204. The two first sensor elements 201, 202 and the two further sensor elements 203, 204 are each formed by an electrode. The four sensor elements 201, 202, 203, 204 or electrodes for detecting a blood circulation signal are disposed for example next to one another on an arm 51, especially a wrist, of the patient 15. As an alternative the four sensor elements 201, 202, 203, 204 can also be disposed on a finger or leg or the neck of the patient 15.

In an operating position of the sensor unit 200 on the arm 51 of the patient 15 the two first sensor elements 201, 202 are formed by the two outer sensor elements 201, 202, between which the two second sensor elements 203, 204 are disposed. An alternating current is applied between the two first sensor elements 201, 202 for detection and/or measurement of the blood circulation signal, for example an alternating current with a current strength of appr. 90 to and a frequency of appr. 100 kHz. As a result of the alternating current applied to the two first or outer sensor elements 201, 202, a voltage is present at the two second sensor elements 203, 204, which is measured for detecting a blood circulation signal. For this purpose the sensor unit 200 has a power supply unit 205 and a voltage detection unit, for example a voltmeter. The voltage present at the two second sensor elements 203, 204 is dependent in this case on physiological parameters of the blood circulation of the patient 15, such as especially dependent on a pulse frequency of the blood circulation.

The sensor unit 200 also has a demodulation unit 207, especially a phase-sensitive demodulation unit 207. By means of the demodulation unit 207 a high signal quality of the detected voltage signal or the detected blood circulation signal is obtained, since especially interferences, for example with a signal of the gradient pulse unit 17, are suppressed.

Furthermore the sensor unit 200 features the data sending unit 28 with the antenna elements 27 of the data transmission unit 25 for wireless and/or cordless data transmission to the data evaluation unit 24 (FIG. 1).

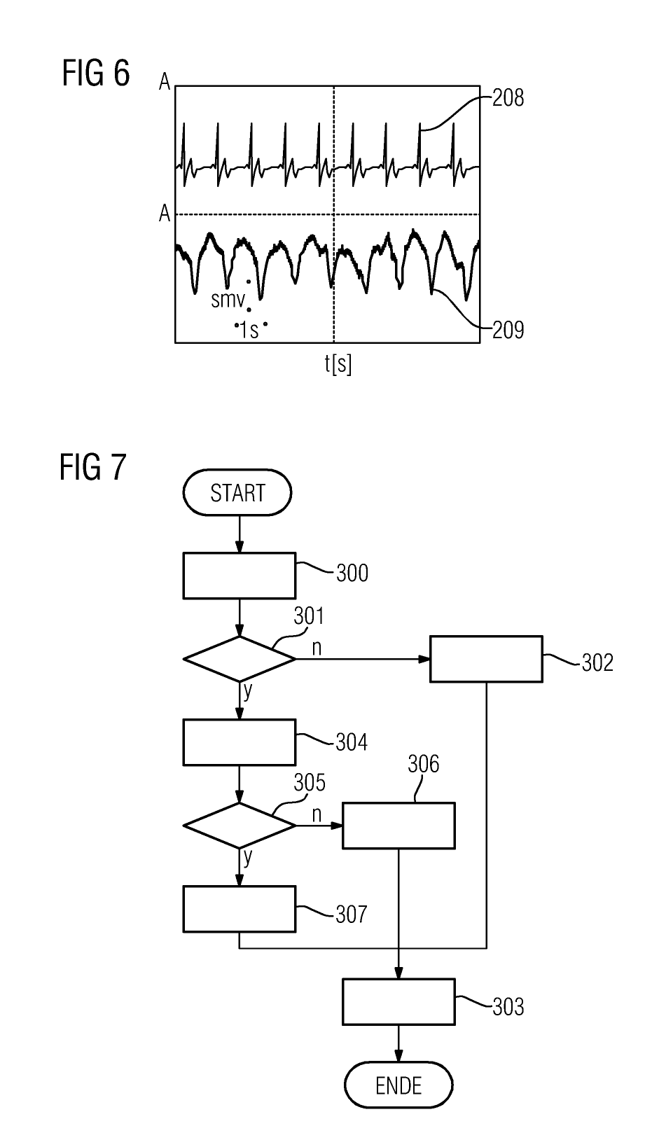

In FIG. 6 an amplitude A of a detected EKG signal 208 of the patient 15 and an amplitude A of a blood circulation signal detected by means of the sensor unit 200, especially by means of the two second sensor elements 203, 204, which is formed here by the voltage signal 209, are plotted over the time t for comparison. Signal maxima or signal minima of the detected voltage signal 209 correlate here with the pulse frequency of the patient 15. By comparison with the EKG signal 208 it is evident that the detected voltage signal 209 correlates with the EKG signal 208 or a cardiac cycle of the patient 15, so that from the detected voltage signals 209 a direct deduction can be made about the cardiac cycle and/or a heart frequency of the patient 15.

FIG. 7 shows an inventive method for detecting the cardiac cycle of the patient 15 for generating a trigger signal for a medical imaging examination, especially for magnetic resonance examination on the patient 15. After preparation of the patient 15, especially fitting the sensor unit 50, 100, 200 and if necessary further sensor units, for example the reference sensor unit 30 to the patient 15, the method for detecting the cardiac cycle of the patient 15 is started.

Initially, in a first method step 300, the blood circulation signal on the patient 15 is detected by means of the sensor units 50, 100, 200 described above before the medical imaging examination, especially the magnetic resonance examination. It is then determined in an interrogation 301 whether reference signals are also being detected. Provided no reference signals are detected, in a further method step 302 correction values are read out from a database on the basis of the detected blood circulation signal. By means of the correction values read out from the database and the blood circulation signal detected by means of the sensor units 50, 100, 200, a trigger signal for the imminent magnetic resonance examination is generated by means of the data evaluation unit 24 in a further method step 303. The correction value includes a delay of the detected blood circulation signal, especially of the pulse signal, to the cardiac cycle of the patient, so that by means of the correction value a delay of the detected blood circulation signal can be taken into account in the determination of the trigger signal.

However if the result of the interrogation 301 is that a reference signal is to be detected, the reference signals are detected in a further method step 304. Subsequently it is established in a further interrogation 305 whether a correction value for the blood circulation signal detected by means of the sensor unit 50, 100, 200 can be calculated correctly. If the response to the interrogation 305 is “no”, in a further method step 306 a correction value is read out from the database based on the detected reference signal. The reference signal for example comprises a blood pressure signal and/or an EKG signal, which is preferably detected before the medical magnetic resonance examination on the patient. For this purpose the patient 15 can likewise still be in a preparation phase for the magnetic resonance examination, such as in a waiting room for the magnetic resonance examination for example.

By means of the correction value read out from the database and the blood circulation signal detected by means of the sensor units 50, 100, 200, a trigger signal for the imminent magnetic resonance examination is generated by means of the data evaluation unit 24 in the further method step 303. The correction value likewise comprises a delay of the detected blood circulation signal, especially of the pulse signal to the cardiac cycle of the patient, wherein here by means of the reference measurement especially exact correction values are available for the determination of the trigger signal.

If the result of the interrogations 305 is that a correction value for the blood circulation signal detected by means of the sensor unit 50, 100, 200 can be computed directly from the reference signal, in a further method step 307 a correction value is computed by the data evaluation unit 24 from a comparison of the detected blood circulation signals and the reference signals. The reference signal can be detected here by means of a navigator measurement by means of the magnetic resonance device, so that from the navigator measurement a heart movement can be detected and thus a delay of the blood circulation signal, for example a pulse signal, to the cardiac cycle of the patient can be determined By means of the correction value determined in the method step 307 and the blood circulation signal detected by means of the sensor unit 50, 100, 200, a trigger signal for the imminent magnetic resonance examination is generated by means of the data evaluation unit 24 in the further method step 303.

The correction values determined in the method steps 202, 306 and 307 are the same for the entire subsequent magnetic resonance examination on the patient 15.

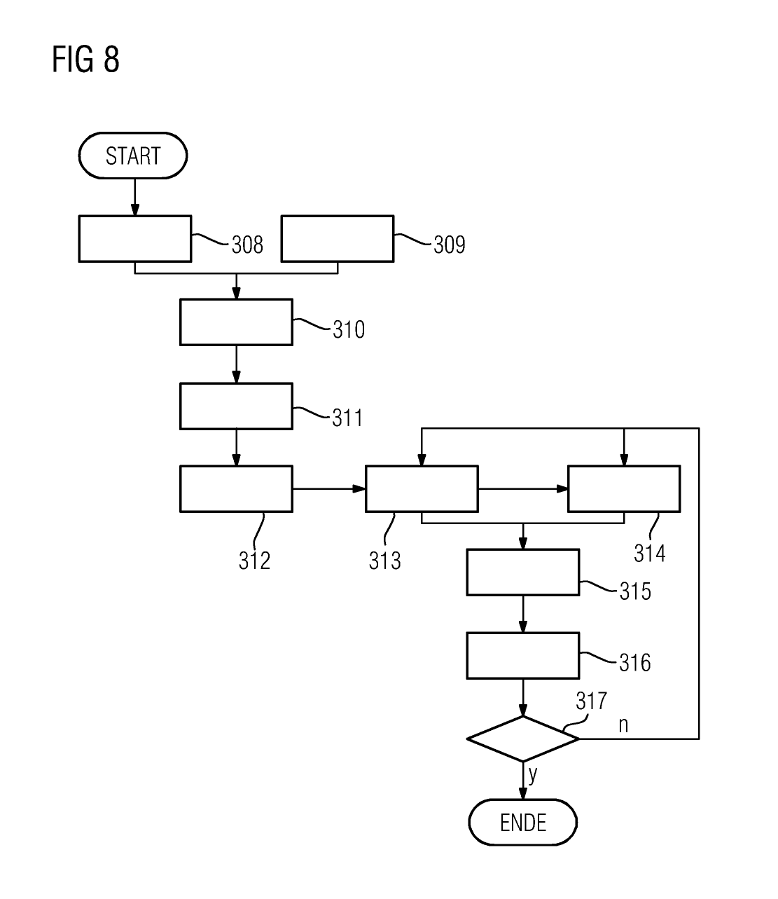

FIG. 8 shows an alternate method to FIG. 7 for detection of a cardiac cycle of a patient 15 for generating a trigger signal for the magnetic resonance examination on the patient. Here too the blood circulation signal, especially a pulse signal, is detected on the patient 15 in a first method step 308 by means of one of the sensor units 50, 100, 200 described above before the medical imaging examination, especially the magnetic resonance examination. The blood circulation signal is detected here before the magnetic resonance examination on the patient 15. At the same time, in a further method step 309, a reference signal is detected, wherein the reference signal is preferably formed by a blood pressure signal. In a further method step 310 a correction value is calculated initially on the basis of the reference signal and/or read out from a database and in a further method step 311 a trigger signal for the medical magnetic resonance examination is generated by means of the data evaluation unit 24.

Subsequently, in the method step 312, the medical magnetic resonance examination is started. In addition, in the method steps 313 and 314, a pulse signal and also reference signal are detected during the medical magnetic resonance measurement. On the basis of the pulse signals and reference signals detected in the method steps 313, 314, in a further method step 315 a correction value is determined, wherein the correction value is determined in a similar way to method step 310. Subsequently, in a further method step 316, a trigger signal is generated on the basis of the correction value and the pulse signal detected in the method step 313, so that individual magnetic resonance measurements are always detected and/or recorded during an identical position within the cardiac cycle of the patient 15. The method steps 313-316 are executed until such time as an abort criterion 417 of the method is present. The abort criterion 317 can for example be an ending of the magnetic resonance measurement.

1. A medical imaging device, comprising:
a sensor unit for detecting a physiological signal, and
a data evaluation unit which, on the basis of the detected physiological signals, determines a trigger signal for a medical imaging examination on a patient (15),
wherein the sensor unit comprises at least one sensor element which is designed for detection of at least one blood circulation signal.

2. The medical imaging device as claimed in claim 1,
wherein the at least one sensor element, for detecting the blood circulation signal, is disposed on an arm and/or the neck and/or a leg of the patient.

3. The medical imaging device as claimed in claim 1,
wherein the at least one sensor element comprises a pulse sensor element by means of which a pulse signal is able to be detected.

4. The medical imaging device as claimed in claim 1,
wherein the at least one sensor element comprises a blood pressure sensor element by means of which a blood pressure signal is able to be detected.

5. The medical imaging device as claimed in claim 1,
wherein the at least one sensor element comprises an optical sensor element.

6. The medical imaging device as claimed in claim 4,
wherein the optical sensor element comprises a laser unit and a laser detector unit,
wherein the laser unit sends out a laser signal and the laser detector unit detects the laser signal reflected on the patient.

7. The medical imaging device as claimed in claim 1,
wherein the sensor unit has at least two first sensor elements and at least two further sensor elements, wherein a voltage is able to be detected between the at least two further sensor elements, during a contact with the patient and an alternating current flowing between the at least two first sensor elements.

8. The medical imaging device as claimed in claim 1,
wherein the sensor unit comprises a data transmission unit with a least one antenna element.

9. The medical imaging device as claimed in claim 1,
wherein a reference signal is able to be detected by means of the sensor unit, wherein the reference signal is embodied differently to the physiological signal.

10. The medical imaging device as claimed in claim 1, further comprising:
a further sensor unit by means of which a reference signal is able to be detected.

11. The medical imaging device as claimed in claim 10,
wherein the further sensor unit comprises an EKG unit.

12. The medical imaging device as claimed in claim 10,
wherein the further sensor unit comprises a blood pressure sensor unit.

13. The medical imaging device as claimed in claim 10,
wherein the further sensor unit is embodied at least in some cases in one piece with a detector unit.

14. A method for detecting a cardiac cycle of a patient for generating a trigger signal for a medical imaging examination on the patient, the method comprising:
detecting a physiological signal on the patient,
determining a correction value for the physiological signal,
evaluating the physiological signal together with the correction value, wherein by means of the correction value a time delay of the physiological signal to the cardiac cycle of the patient is determined, and
generating a trigger signal depending on the detected physiological signal and the determined time delay by means of a data evaluation unit.

15. The method as claimed in claim 14,
wherein the correction value is read out from a database.

16. The method as claimed in claim 14,
wherein a reference signal is detected on the patient for determining the correction value.

17. The method as claimed in claim 16,
wherein the reference signal is detected before the medical imaging measurement.

18. The method as claimed in claim 16,
wherein the reference signal is detected during the medical imaging measurement.

19. The method as claimed in claim 16,
wherein the reference signal comprises an EKG signal and/or a blood pressure signal.

20. The method as claimed in claim 14,
wherein the reference signal comprises a signal of a navigator measurement of the medical imaging device.

21. The method as claimed in claim 14,
wherein the physiological signal is a first blood circulation signal.

22. The method as claimed in claim 21,
wherein a further blood circulation signal, which is embodied differently to the first blood circulation signal, is detected at least in some cases during the medical imaging examination on the patient and that, depending on the further blood circulation signal, a change in the time delay to the cardiac cycle of the patient is determined.
